(19) United States
(12) Patent Application Publication
(10) Pub. No.: US 2023/0172491 A1
(43) Pub. Date: Jun. 8, 2023

(54) SYSTEM AND METHOD FOR MOTION ANALYSIS INCLUDING IMPAIRMENT, PHASE AND FRAME DETECTION

(71) Applicant: Stephen GROSSERODE, SANTA MONICA, CA (US)

(72) Inventor: Stephen GROSSERODE, SANTA MONICA, CA (US)

(21) Appl. No.: 17/998,035

(22) PCT Filed: May 5, 2021
(86) PCT No.: PCT/US2021/030979

§ 371 Date: Nov. 5, 2022

Related U.S. Application Data
(60) Provisional application No. 63/020,540, filed on May 5, 2020.

(51) Int. Cl. A61B 5/11; G06T 7/246; G06V 10/764; G06V 40/20; G06V 20/40; G06T 11/00; G06T 7/00; A61B 5/00

(52) U.S. Cl. CPC A61B 5/1128; A61B 5/0004; A61B 5/0077; A61B 5/742; A61B 5/7282; G06T 7/0012; G06T 7/246; G06T 11/00; G06V 10/764; G06V 20/42; G06V 40/25; A61B 2503/10; G06T 2200/24; G06T 2207/10016; G06T 2207/30004; G06T 2207/30196; G06T 2207/30221; G06V 2201/03

(57) ABSTRACT

Among other things, embodiments of the present disclosure can detect a movement impairment within one of at least one critical phase or frame and/or instance in time of body movement, based at least on movement analysis data obtained by data captured via a camera coupled to a computer system or input into the computer system. The movement analysis data may include at least one critical phase or frame and/or instance in time of body movement. Information related to the detected movement impairment is displayed by the computer system on the display screen.

This application claims the benefit of priority of U.S. Pat. Application Ser. No. 63/020,540, filed May 5, 2020, the contents of which are hereby incorporated by reference in their entirety.

### I. FIELD

Example aspects described herein generally relate to motion analysis, and more specifically relate to systems and methods for determining and analyzing motion of a subject, as well as analyzing movement data obtained therefrom.

### II. BACKGROUND

Motion analysis is an important part of the discipline of biomechanics, and can be associated with various applications such as, for example, sports medicine, physical therapy, balance assessment, force sensing measurement, sports science training, physio analysis, and fitness equipment operation, etc. Motion analysis is typically performed based on images, in which a system captures a sequence of images of a subject (e.g., a human being) when the subject is engaged in a specific motion. The system can then determine, based on the sequence of images, the positions of various body segments of the subject at a given time. Based on the positions information, the system can then determine a motion and/or a posture of the subject at that time.

### BRIEF DESCRIPTION OF THE DRAWINGS

The embodiments herein are illustrated by way of example and not by way of limitation in the figures of the accompanying drawings in which like references indicate similar elements. It should be noted that references to “an” or “one” embodiment in this disclosure are not necessarily to the same embodiment, and they mean at least one. Also, in the interest of conciseness and reducing the total number of figures, a given figure may be used to illustrate the features of more than one embodiment, and not all elements in the figure may be required for a given embodiment.

FIG. 1A is a diagram depicting a system including a standalone client on which embodiments of the invention can be implemented.

FIG. 1B is a network diagram depicting a network system having a client-server architecture configured for exchanging data over a network, on which embodiments of the invention can be implemented.

FIG. 2 shows a flow diagram illustrating a process that can be performed on either one of the systems of FIGS. 1A and 1B, according to an example embodiment.

FIG. 2 (Cont.) shows a flow diagram for illustrating a process that can be performed during or within blocks 213 and 214 of FIG. 2.

FIG. 3 shows a diagrammatic representation of machine, in the example form of a computer system, within which a set of instructions may be executed to cause the machine to perform any one or more of the methodologies discussed herein.

FIGS. 4-24 depict example outputs displayed, for example, on a display screen, according to example embodiments.

### DETAILED DESCRIPTION

Several embodiments are now explained with reference to the appended drawings. Whenever aspects are not explicitly defined, the embodiments are not limited only to the parts shown, which are meant merely for the purpose of illustration. Also, while numerous details are set forth, it is understood that some embodiments may be practiced without these details. In other instances, well-known circuits, structures, and techniques have not been shown in detail so as not to obscure the understanding of this description.

The inventor herein has found that current technologies provide various ways of performing image-based motion analysis. One approach is by tracking a motion of markers emitted by the subject. For example, the subject can wear a garment that includes a number of markers. The markers can be passive reflector (e.g., with VICON™ system) or active emitter of visible light or infra-red light (e.g., with PhaseSpace™ system). The system can then use a plurality of cameras to capture, from different views or vantage points, a sequence of images of the markers when the subject is in a motion. Based on the sequence of images, as well as the relative positions between each camera and the subject, the system can determine the motion of the subject by tracking the motion of the markers as reflected by images of the markers included in the sequence of images.

Another approach is by projecting a pattern of markers on the subject and then tracking the subject’s motion based on images of the reflected patterns. For example, Microsoft’s Kinect™ system projects an infra-red pattern on a subject and obtains a sequence of images of the reflected infra-red patterns from the subject. Based on the images of the reflected infra-red patterns, the system then generates depth images of the subject. The system can then map a portion of the depth images of the subject to one or more body parts of the subject, and then track a motion of the depth images portions mapped to the body parts within the sequence of images. Based on the tracked motion of these depth images portions (and the associated body parts), the system can then determine a motion of the subject.

The inventor herein has found that there are disadvantages for both approaches. With the VICON™ system, the subject will be required to wear a garment of light emitters, and multiple cameras may be required to track a motion of the markers in a three-dimensional space. The additional hardware requirements substantially limit the locations and applications for which the VICON™ system is deployed. For example, the VICON™ system is typically not suitable for use at home, outside or in an environment with limited space.

On the other hand, the Kinect™ system has a much lower hardware requirement (e.g., only an infra-red emitter and a depth camera), and is suitable for use in an environment with limited space (e.g., at home). The accuracy of the motion analysis performed by the Kinect™ system, however, is typically limited, and is not suitable for applications that demand high accuracy, variability in environment, and highly dynamic movements of motion analysis.

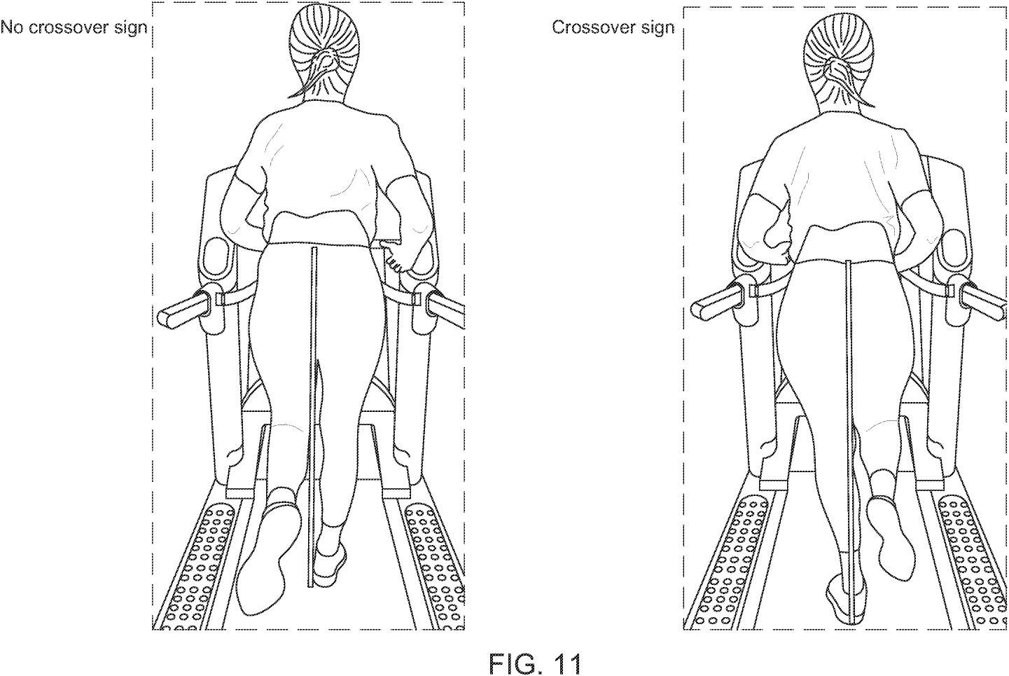

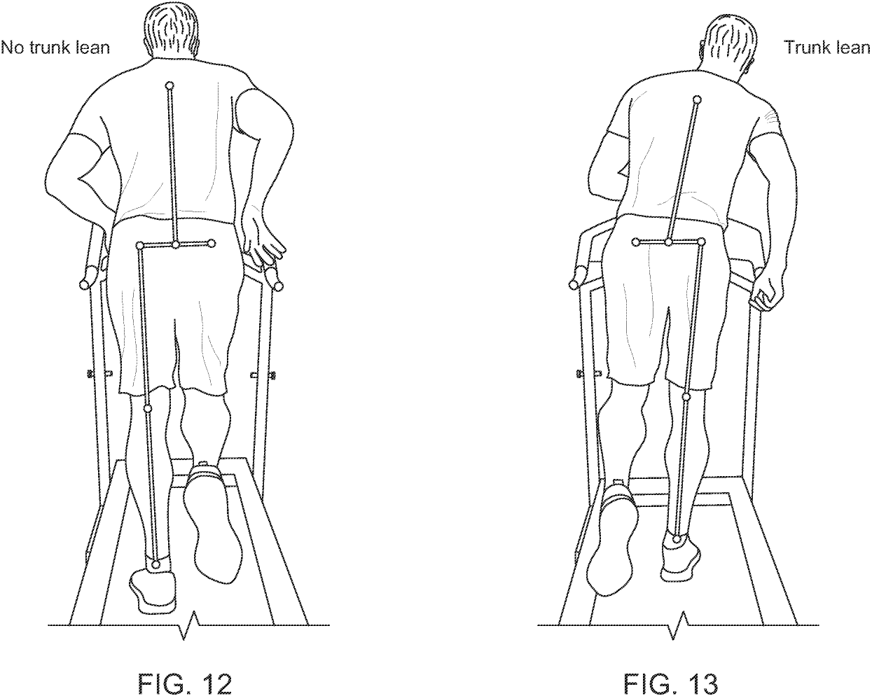

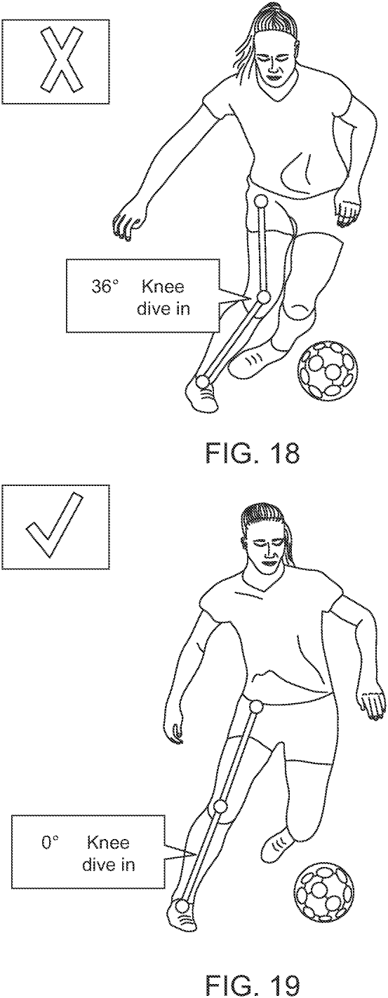

The disclosure herein addresses the foregoing problems of current motion analysis systems by providing a computer-implemented system that obtains movement analysis data captured via a camera coupled to the computer system or input into the computer system, and performs highly accurate motion analysis, without requiring substantial hardware requirements. The computer system includes a display screen coupled to the computer system. This system is not limited to capturing data from a computer system in the present moment. The system is also capable of receiving input videos and/or still images that were previously captured and then analyze the input videos and/or still images by overlaying the movement analysis data on top of the video and/or frames. The movement analysis can be, for example, displacement and orientation of the segments of the body, joint angles, to recognize if they are within normal parameters, etc. The movement analysis data includes at least one critical phase or at least one image frame of body movement. This can be defined as phase detection and frame detection, respectively. The phase detection can provide detection of specific phases of a particular movement that can be predetermined based on empirical research and/or expert opinion. The frame detection can automatically capture any frame decided on by a user. This information can include all associated data, such as kinematic data, that corresponds to that moment in time. According to one aspect, the computer system can detect a movement impairment within one of the at least one critical phase or image frame of body movement, based at least on the obtained movement analysis data. A movement impairment can be defined as an abnormal movement alignment such as a joint’s angle during a moment in time that is outside of normal parameters. A comparison of normal and outside of normal parameters are shown, for example, in FIGS. 11, 12, 13, 17, 18 and 19. Such movement impairments have been shown to be associated with many musculoskeletal conditions such as patellofemoral pain syndrome, ACL injuries, etc. as well as neurological conditions such as Parkinson’s disease. The methods of movement analysis, impairment detection, phase detection, and frame detection disclosed herein aid in the classification/determination and treatment of medical conditions such as orthopedic and neurological conditions. The methods of phase detection and frame detection disclosed herein provide an improvement on conventional motion analysis systems in that these detections can be performed using algorithms executed on, for example, a compact portable device or server connected thereto, without excessive and encumbering hardware. After making a detection, the computer system can then display on the display screen, information related to the detected movement impairment and/or the detected phase or image frame.

By improving the computer technology of motion analysis systems, the embodiments disclosed herein can provide the advantageous effects of having a portable, versatile, easy-to-use computer system that accurately analyze motion data without requiring bulky, burdensome hardware. The embodiments disclosed herein can also provide the advantageous effect of allowing for remote analysis such that the analysis and applications thereof can be provided in a case where the practitioner and the client/patient are in different, separate and/or remote locations. Another advantageous effect includes the ability to analyze the runner, athlete etc. in their natural environment such as outside, on field, on court etc.

According to another aspect, the movement analysis data can be captured via the camera, or can be a previously captured video or frame, for a plurality of moving bodies or subjects, and a movement impairment can be detected for each of the plurality of moving bodies or subjects.

According to yet another aspect, the movement analysis data can be captured using markerless tracking. By virtue of this aspect, the computer system can detect anatomical landmarks without requiring a practitioner to manually find the landmark and then manually place markers on the body as is the case in conventional known systems. The computer system also allows for numerous detections based on points on the body in relation to one another and the angles, distance, etc. between them. These detection parameters are capable of being modified by the user. This allows a user to modify the placement of a virtual marker similar to how they would modify marker placement with actual markers.

The detection performed by the computer system can involve numerous different detections being performed synchronously or asynchronously, and individually or in combination with other detections. The detections may include one or more of detecting a direction in which a body is moving, detecting a running cadence and stride length of the moving body, detecting a center of mass displacement of the moving body, and detecting and labeling a type of joint or body part of the static or moving body.

According to an additional aspect, the computer system can include a server, where the obtained movement analysis data is transmitted to the server, and the one or more detections are performed in near real-time at the server. The obtained movement analysis data is capable of being integrated with other platforms.

In other aspects, the detecting is performed at the computer system connected to the display screen in real-time.

According to another aspect of the computer system, the computer system can perform the detection by processing and/or analyzing and comparing the processed movement analysis data with normative values, historical data, or the process movement data itself, or a combination of these comparisons. The computer system can also use manually input text data in the foregoing detections.

In yet another aspect, the computer system can predict a likelihood that a specific injury will occur based at least on analysis of the movement data. The advantageous effect of this is to provide interventions based on which injury is likely to occur in order to prevent that injury altogether.

According to another aspect, the computer system can display one or more of displaying a classification or determination and/or interpretation of each datapoint such as joint angles, displaying a classification or determination of the impairment, displaying one or more highlighted sections of the movement analysis data which are deemed red flags or outliers, displaying exam recommendations, and displaying impairment corrections and/or treatment. Displaying exam recommendations can include providing an impairment ranking for a diagnostic hypothesis list and/or prediction of an injury. Displaying exam recommendations can include providing an impairment ranking for a diagnostic hypothesis list and/or prediction of an injury. Displaying treatment recommendations can include correction exercises, corrective movements, activity modifications, product recommendations, and any other known recommendations for treatment of that condition.

The computer system can also detect at least one of the critical phases of specific movements, as determined by research or expert opinion, within the movement analysis data. According to this aspect, a specific frame, based on research or expert opinion which indicates such specific frame to be a phase of that movement, can be detected within a critical phase of the body movement based on angle or point detection. The computer system can also detect a specific frame decided on by the user.

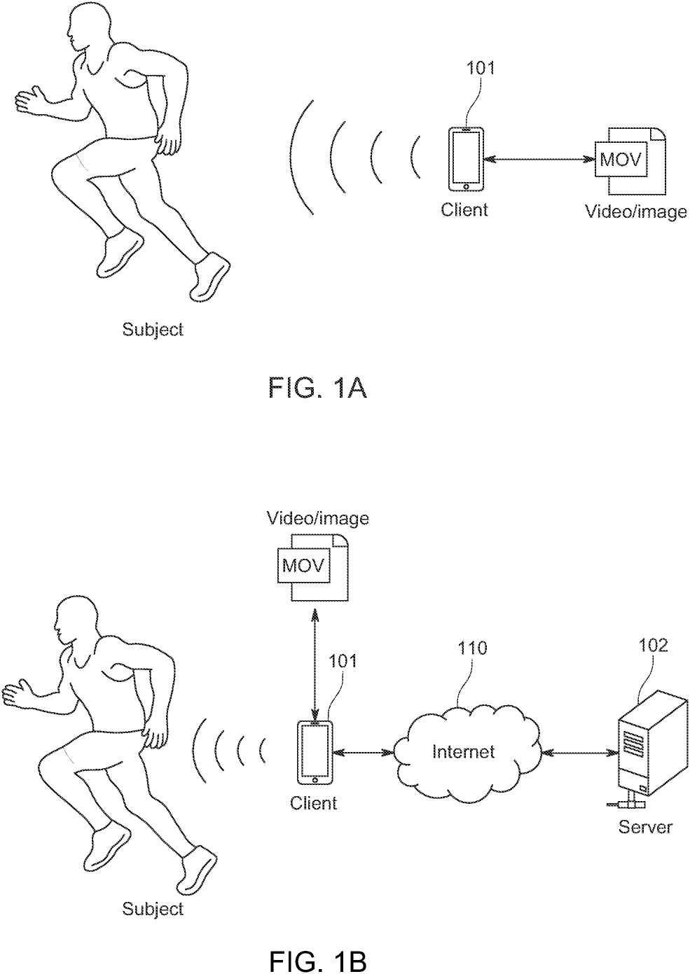

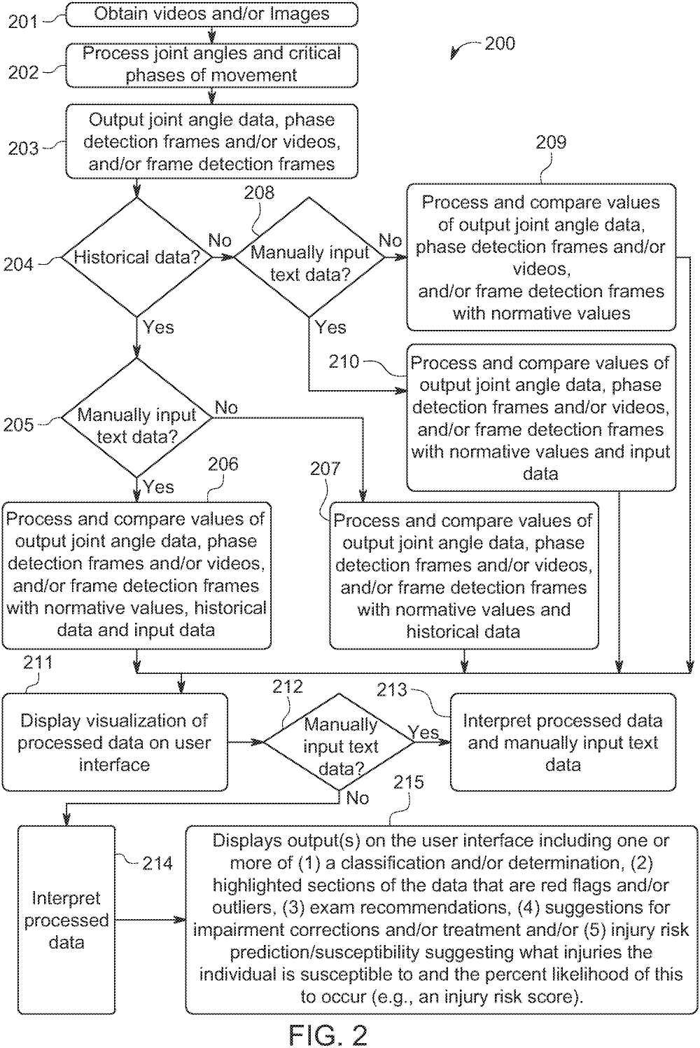

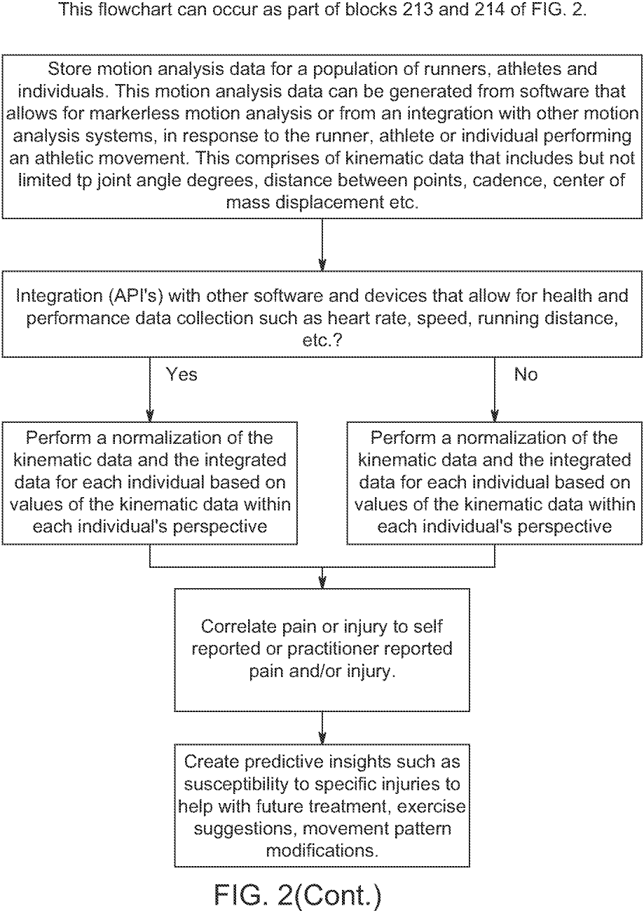

FIG. 1A illustrates a computer system including a standalone client 101 on which an example embodiment can be implemented. As shown in FIG. 1A, a subject using the client 101 as an image/video capturing device can take video or pictures. A user such as the subject can also input video and/or images into the client 101. In this embodiment, the image/video is then processed using the standalone client 101, as described, for example, in detail below in connection with FIG. 2.

FIG. 1B illustrate a network system having a client-server architecture configured for exchanging data over a network, on which another example embodiment can be implemented. As shown in FIG. 1B, the client 101 communicates with the server 102 by transferring data via Internet 110 (or another network such as a LAN). In this embodiment, the user takes video or pictures on any image/video capturing device such as client 101, the client 101 send the image/video to the server 102 via Internet 110, and the processing is performed at the server 102. A user such as the subject can also input video and/or images into the client 101. A comparison is made by the computer system, either on the client 101 or the server 102 (as discussed in more detail in connection with FIG. 2), and visualizations are displayed on the client 101.

FIG. 2 shows a flow diagram illustrating a process that can be performed on either one of the systems of FIGS. 1A and 1B, according to an example embodiment. The following embodiments may be described as a process 200, which is usually depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a procedure, etc.

Process 200 may be performed by processing logic that includes hardware (e.g. circuitry, dedicated logic, etc.), software (e.g., embodied on a non-transitory computer readable medium), or a combination thereof, on either the client 101 and/or the server 102 of FIGS. 1A and 1B. In one example embodiment, process 200 is executed by a software driver executing on a CPU or a GPU of the client 101 and/or server 102.

Referring to FIG. 2, at block 201, the computer system obtains videos and/or still images of a subject or subjects for analysis. The videos and/or still images can be obtained using any type of camera device, for example, included in the client 101. The videos and/or still images can also be obtained by manual input by the subject and/or a user. The videos and/or still images are then processed at the client 101, or sent to the server 102 for processing there, as described in more detail below.

The collection of movement data can be performed using a markerless system. This is in contrast to and an improvement over commonly known methods that would require markers that were placed on specific areas of the body (e.g., landmarks) to be able to detect kinematic data such as joint angles, etc. The system and processes disclosed herein automate the process of collecting movement analysis data. As discussed above, conventional systems require specialized hardware. The collection and processing of markerless motion analysis data is compatible with any camera system, including phones and tablet devices.

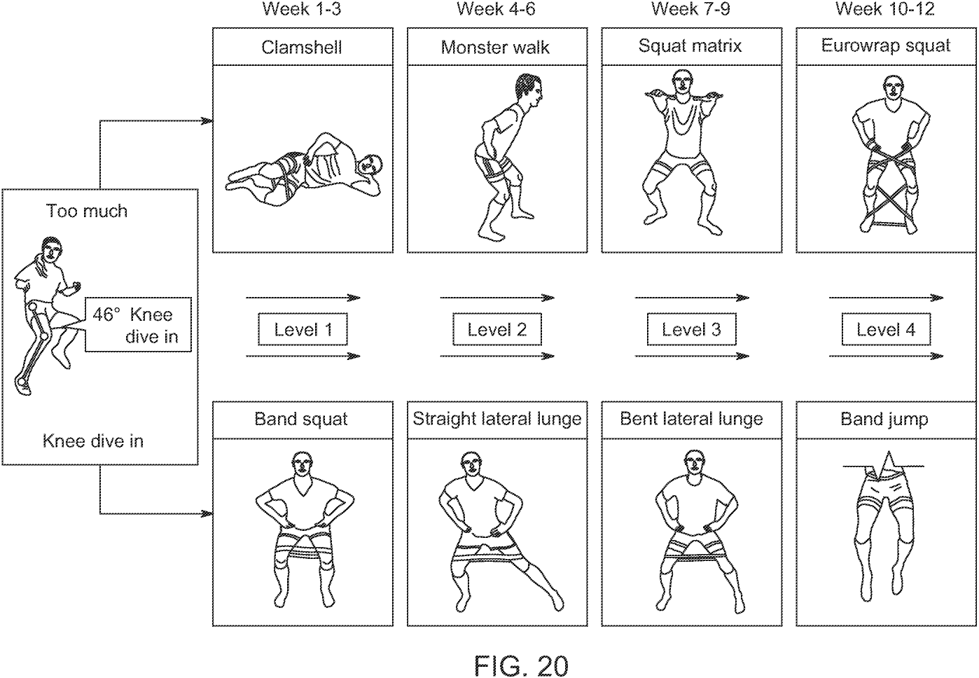

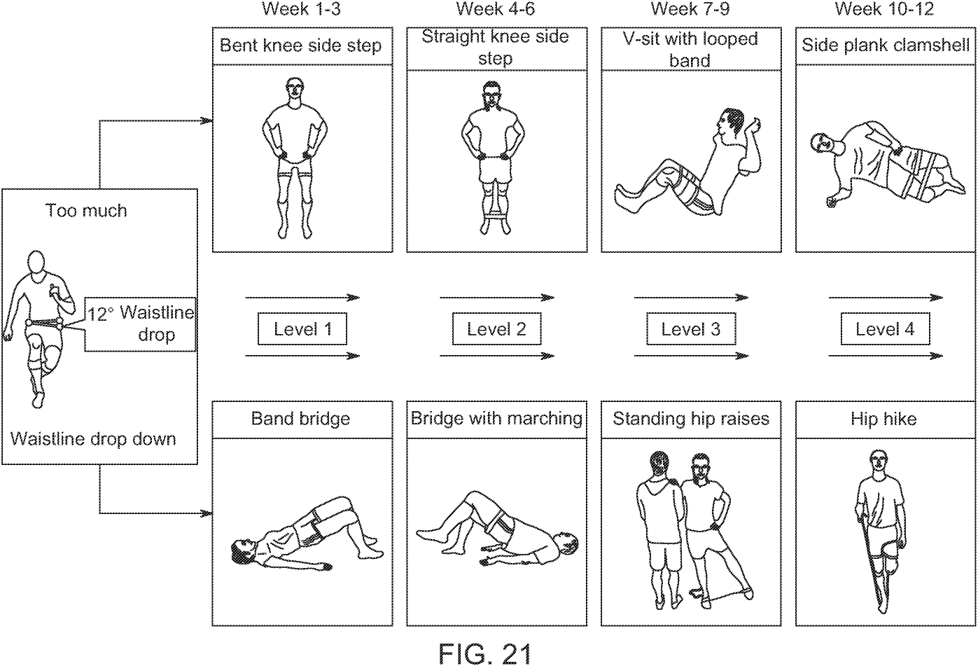

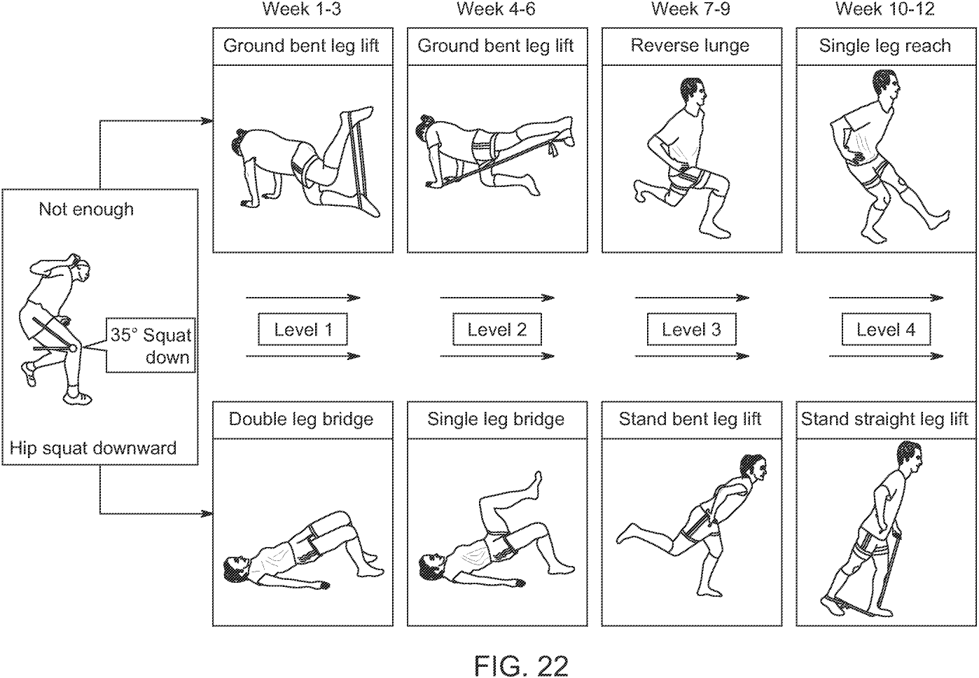

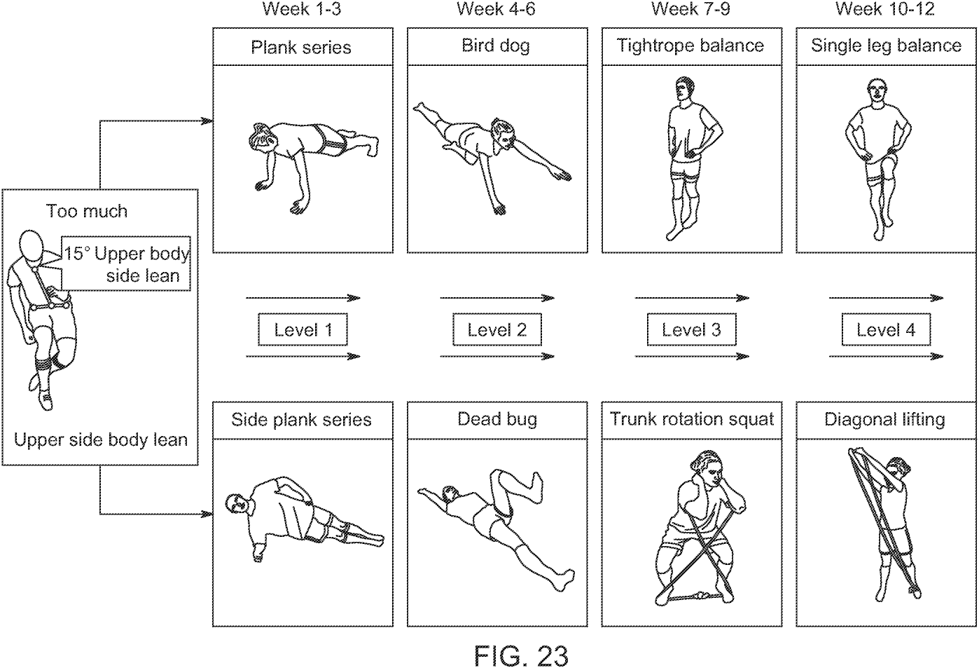

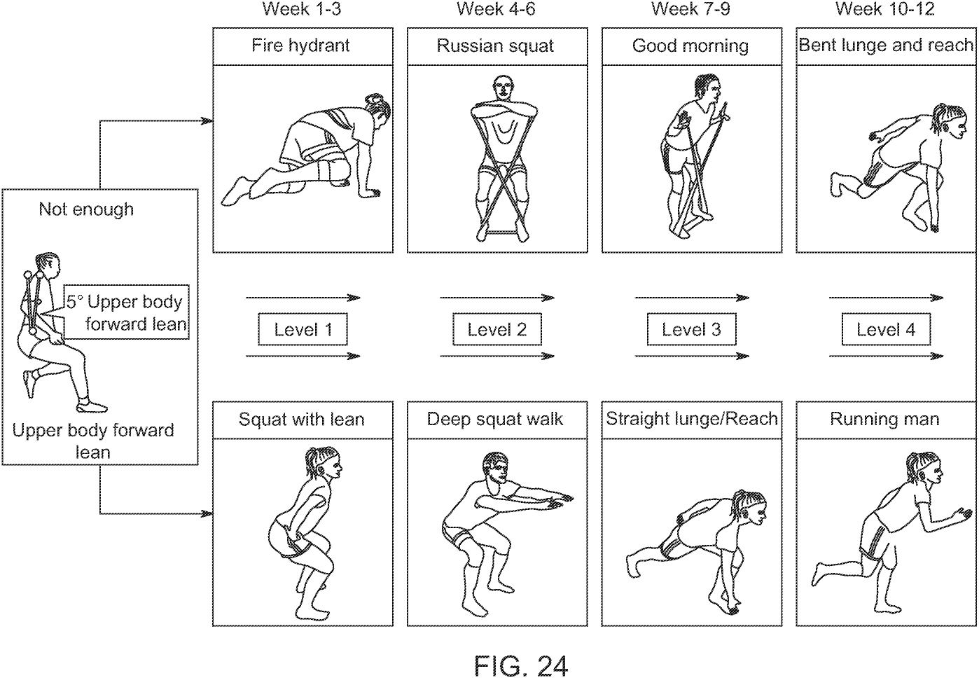

Moreover, automatic snapshot(s) of specific/critical moments (“phases”) or image frames during gait, running and other movements can be collected. Subjects can be individuals or groups of people simultaneously, and the data transfer can occur simultaneously from all subjects. The disclosure herein is not limited to markerless motion analysis, and is also capable of processing any image or video. A user and/or subject can upload any video to the system and processes can analyze the video. For example, a regular video can be processed to produce a markerless motion analysis video, that is comparable to markerless motion analysis that requires hardware equipment such as Vicon™. The data from this processed video can be used to create predictions about the stresses that are placed on the human body during this movement that can eventually lead to medical conditions. These predictions allow for guidance with recognition of the stresses that a certain movement pattern places on the subject’s body and make suggestions for corrective measures in order to prevent and treat injury. Examples of determinations and corrective measures are shown in FIGS. 20-24. As shown in FIGS. 20-24, the movement determination and/or classification is shown on the left, and the arrows points to suggested progression of corrective measures.

Also, for markerless motion analysis with groups of people, data can be gathered for each individual by identifying individual people in the frame and assigning them their own data. Data from the groups of people can be segmented so each individual person’s data can be collected and added to over time. The technology can recognize the individual and place the correct kinematic data into that user’s profile. This includes, for example, videos, images, frames, kinematic data, etc.

At block 202, the computer system processes joint angles and critical phases of movement known as phase detection or the frame desired by the user known as frame detection using the obtained videos and/or images. This computer system can capture data in the present moment to perform data analysis. The computer system can also take videos that were previously captured and analyze them by overlaying the data on the video and/or frames.

As part of the processing, the computer system automatically pulls data from gait/running analysis, movement/motion analysis and inputs the data into a table, graph or any type of data display and/or electronic medical record. The automatically inputted data can also be integrated with an online platform that allows the practitioner in a healthcare, fitness or sports setting to manipulate and/or add to the data. This also allows an end user such as the subject, patient, athlete or fitness person to view the data and potentially manipulate or add to the data.

At block 203, the computer system outputs joint angle data, phase detection frames and/or videos, and/or frame detection frames, which is described in more detail below.

At block 204, the computer system determines whether historical data is available. It is noted that in some embodiments block 204 (as well as blocks 205-208 and 210) are considered optional. In other embodiments, process can flow from block 203 directly to block 209.

At block 205 (if “yes” at block 204), the computer system determines whether text data has been manually input from a practitioner and/or client. At block 206 (if “yes” at block 205), the computer system processes and compares values of output joint angle data, phase detection frames, frame detection frames and videos with the historical data and the input data. At block 207 (if “no” at block 205), the computer system processes and compares values of output joint angle data, phase detection frames, frame detection frames and videos with the historical data. At block 208 (if “no” at block 204), the computer system determines whether text data has been manually input from a practitioner and/or client. At block 209 (if “no” at block 208), the computer system processes and compares values of output joint angle data, phase detection frames, frame detection frames and videos with normative values. At block 210 (if “yes” at block 210), the computer system processes and compares values of output joint angle data, phase detection frames, frame detection frames and videos with normative values and input data.

At block 211, the computer system displays visualization of processed data on a user interface (UI). At block 212, the computer system determines whether text data has been manually input from a practitioner (e.g., objective exam). It is noted that in some embodiments block 212 is considered optional. In other embodiment, the process can flow from block 211 directly to block 214.

At block 213 (if “yes” at block 212), the computer system interprets the processed data and manually input text data. At block 214 (if “no” at block 214), the computer system interprets the processed data.

The computer system through its algorithms and improvement on computer technology allows automatic detection of movement impairments (e.g., the ability to automatically detect whether a person is moving correctly in a manner that can minimize risk for injury, or whether the body is moving in a manner that has been shown to lead to stress, strain, etc.). The computer system also allows for automatically highlighting and displaying sections of the data that are outliers/red flags/alerts, discussed in more detail below.

At block 215, the computer system displays output(s) on the user interface including one or more of (1) a classification and/or determination such as an improper movement of the body, falling out of a normal range suggested parameter, (2) highlighted sections of the data that are red flags and/or outliers, out of the normal limit, within a moderate limit, or within normal limits (3) exam recommendations, (4) suggestions for impairment corrections and/or treatment and/or (5) injury risk prediction/susceptibility suggesting what injuries the individual is susceptible to and the percent likelihood of this to occur (e.g., an injury risk score).

The computer system can display a graphical output to inform the user of this outlier/alert/alarm. In the display, the computer system can automatically label a type of angle as it relates to the anatomy of the subject (e.g., a knee angle, a trunk angle, a hip angle, etc.). The computer system can classify the movement data as a movement determination/classification, and communicate which impairment(s) the subject is demonstrating the most. This movement determination/classification can offer insight and help aid the treatment of medical musculoskeletal and neurological conditions.

In interpreting, the computer system can display the potential causes and penalties of the detected impairments. Penalties may include susceptibility to stress, strain, loading, compression on certain body structures, overuse of certain body structures, compensations, and subsequent alteration of movement, etc. For example, the computer system may display “This combination of impairments have been known to cause XYZ musculoskeletal condition.” In interpreting, the computer system can also display a suspected determination/classification of a musculoskeletal condition, neurological condition, or any other body system condition. For example, the computer system may display “This movement determination/classification is associated with XYZ neurological condition.”

The computer system can also provide an impairment ranking (e.g., based on a severity of impairment). This can be performed by the computer system by comparing to normative values that are in the online repository/cloud server. Once enough data is collected, then the computer system can compare to data collected by users of this technology. An illustrative example of the foregoing is as follows: “XYZ research has shown that during the phase of Midstance the knee angle should be at XYZ degrees. This current knee angle has been known to make runner susceptible to XYZ injuries.”

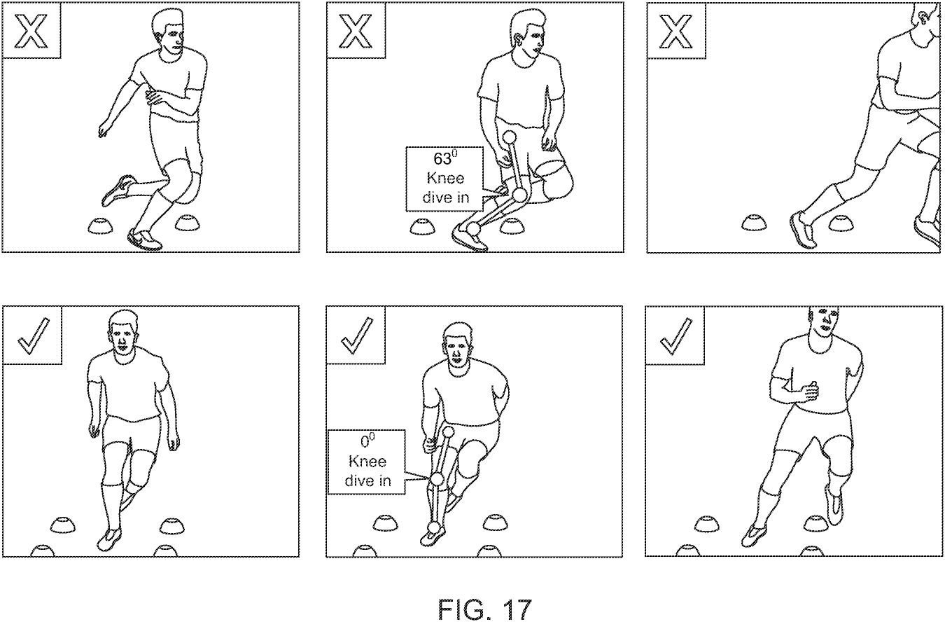

The computer system can also provide recommendations for a physical exam conducted by the practitioner or self-guided exam conducted by the user/client/patient. These recommendations can include how to change movement impairment (e.g., can be used for treatment of current injury or prevention of future injury) such as impairment correction including, for example, exercise, movement, thought, product/device recommendations and other recommendations. The treatment and/or injury prevention suggestions can be for the purpose of guiding exercise prescription in order to correct impairments. Movement determinations can be linked to the specific exercise/corrective measures, as shown, for example, in FIGS. 20-24. Additionally real-time or near real-time feedback can be given following a self-guided exam conducted by the user/client/patient for example, in FIGS. 17-19.

A practitioner and/or subject or client can also guide the recommendations provided by the computer system, by integrating manually input text data (from the practitioner and/or the client such as Rate of Perceived Exertion RPE, pain scale ranking, subjective statements, goals, etc.) combined with results from the movement analysis/impairment detection.

By nature of the computer technology improvements, the computer system disclosed herein provides real-time (or near real-time in the case of sending and receiving data to and from the client 101 and server 102) impairment detection within a single motion of a body. The computer system is capable of detecting impairments at specific phases or frames/instances in time during that movement, and provide recommendations on how to change that impairment. The single motion can also be defined as a critical phase or a single moment in time. The markerless motion analysis can be used with the impairment detection with automatic phase detection and frame detection to detect a single point in a time (moment in time). Markerless motion analysis can be, for example, collecting kinematic data without the use of physical markers placed on the body.

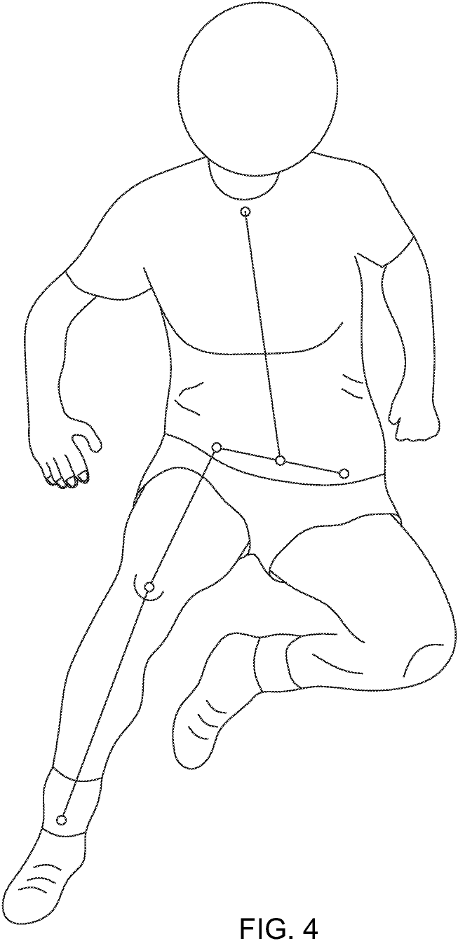

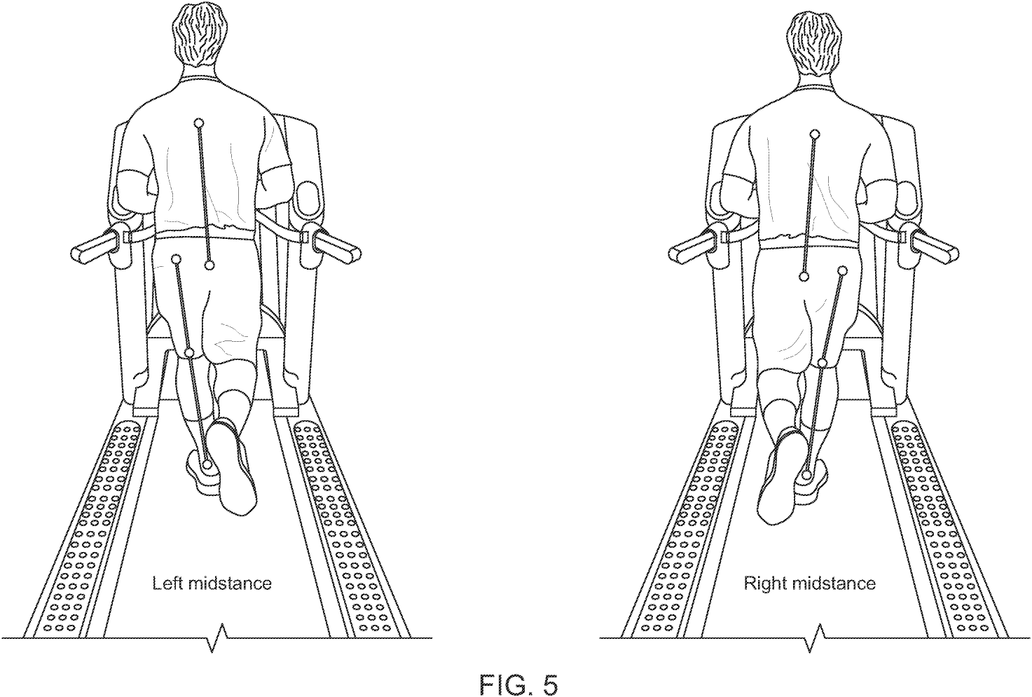

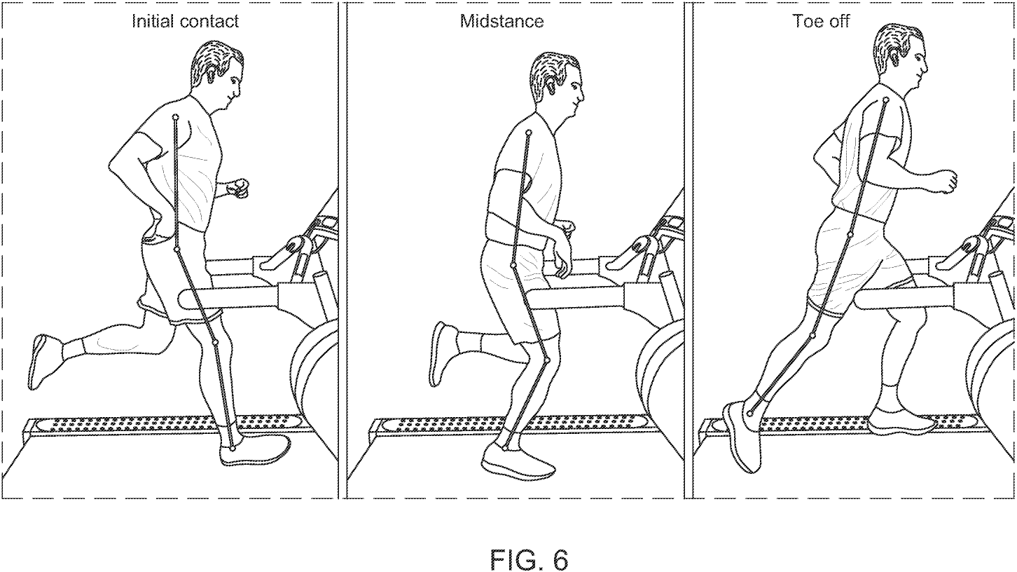

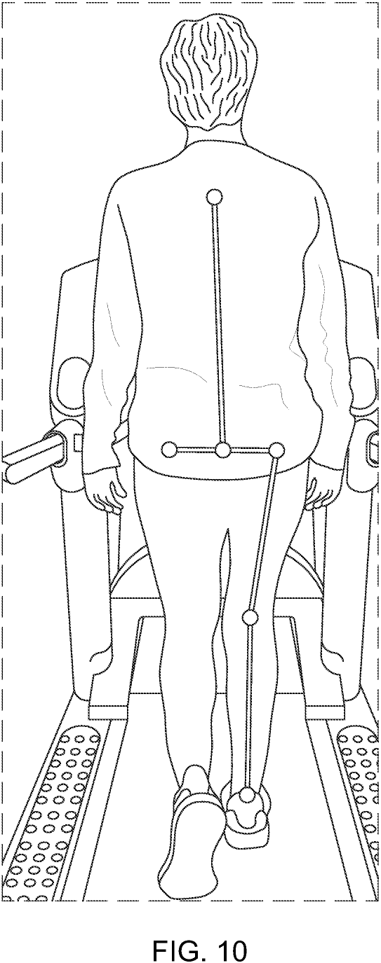

FIGS. 4-24 show examples of subjects and display outputs of the computer system.

For example, with activities of daily living, running, and athletic movements involving the lower extremities including but not limited to gait, running, cutting, jumping, squatting, lateral shuffle etc., and upper extremities including throwing/pitching, shooting a ball, climbing, swimming, serving, swinging, etc., the computer system is looking at the exact moment in time (“phases”) and each individual moment in time (e.g., frames) of that particular movement.

Each body movement goes through a finite amount of critical phases, for example gait can have 8 phases and running can have 8 phases, depending on which group of research is being referenced. In addition to phases, movements can have periods in time where impairments tend to occur. These are typically the moments in time which are viewed critically in order to treat and prevent injuries. For example, during an athletic or running movement, the computer system can detect if there is a certain moment in time where the joint angles fall out of proper range that could put stress on the body. As described above, the computer system can determine the foregoing by comparing this value with normative data OR data that the user decides to input. The result is then to determine if this puts stress on the body, aid in understanding why symptoms might occur, guide intervention and/or prevent injuries.

The proper range can be determined, for example, in various ways: (1) normative data derived from the latest research, (2) with this system through data analysis that can include machine learning, and (3) expert opinion. The computer system then automatically highlights and detects sections of the data that are outliers/red flags to give recommendations. The computer system can automatically highlight sections of the data that are outliers/alerts/alarms and display a graphical output to inform the user of this outlier/alert/alarm. The user can also input and or modify the range based on expert opinion.

FIGS. 11-13, 15, and 17-24 provide examples of visual representations of impairments, according to example embodiments.

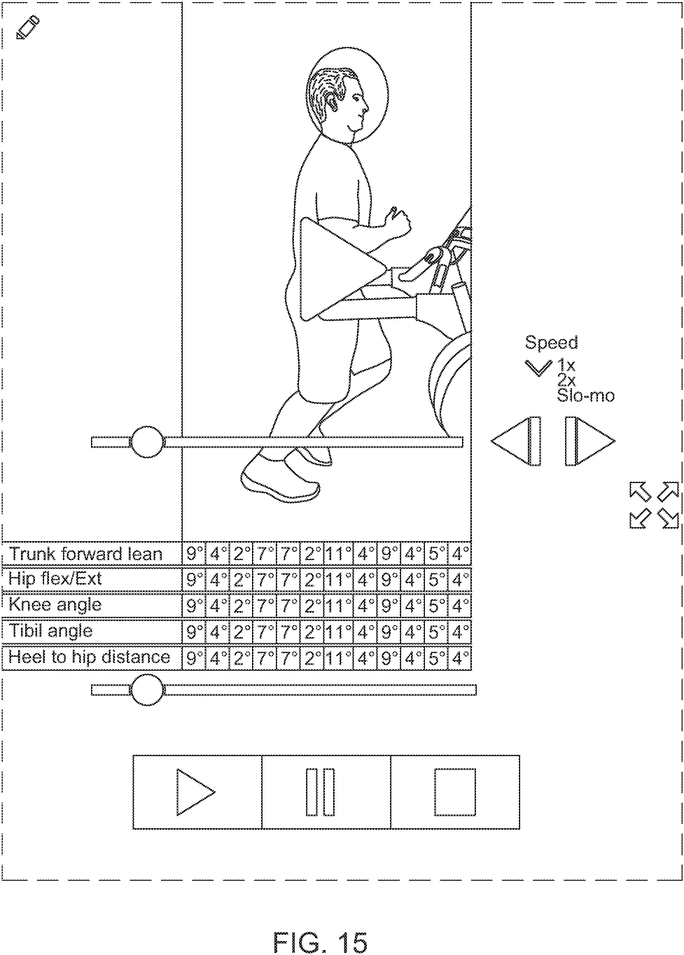

The computer system provides a visual representation to demonstrate what angles are appropriate and which angles are impairments, as shown, for example, in FIG. 15. For every given phase of gait and other movements there are appropriate joint angles. Therefore for a critical phase of that particular movement, the joint angles of the subject will be compared to normative data. Then the computer system will create a visualization to demonstrate whether the joint angle is within the normal limits or if it is outside of normal limits and thus an impairment. This visualization can be represented by a change in color of the angle itself and the numeric value, or be represented as a change in color of the values in the data table, or it may have a label that comes up, or a sound or words that demonstrates if the subject demonstrates an impaired movement. Additionally, a numeric value or score can be given to indicate the individual’s susceptibility to injury and/or level of performance.

With respect to the integration of data, the computer system has the capability to automatically take data from gait/running analysis, movement/motion analysis and input into a table, graph or any type of data display and/or electronic medical record (EMR). Integration (automatic data input) with an online platform or application can allow the individual or practitioner in a healthcare, fitness or sports setting to manipulate and/or add to the data and send the data back and forth between one platform to another. The computer system also allows the end user and/or the patient, athlete or fitness person to view the data. Data can be integrated with the user interface (UI). There can also be integrations such as an application programming interface (API) with other platforms such as electronic medical record (EMR) platforms. The data can be formatted in a manner that allows transfer to all electronic medical records.

With respect to the different detections capable of being performed by the computer system, the following detections can be as follows: (1) Detects the direction the subject is moving (e.g., the software detects which direction the person is running.); (2) Detects when the subject is in stance versus swing (e.g., foot on the ground versus foot in the air); (3) Detects the subjects running cadence (e.g., steps per minute) and stride length; (4) Detects the center of mass displacement (e.g., how high the subject’s body moves up and down during running and athletic movements); (5) Detects the critical phases of specific movements such as gait, running, pitching, a tennis serve, etc.; (6) Detects the anatomical landmarks such as greater trochanter, PSIS, etc., and (7) Detects and tracks the data for any other point on the body the user chooses which can be known as point detection.

A detailed discussion will now be provided to frame detection of the specific phases of gait, running & other movements as well as frame detection of specific moments in time chosen by the user. By way of background, movements such as gait, running, pitching, a tennis serve, squatting, weightlifting etc., all have specific phases of movement. For each phase or point in time of these movements, the subject can have proper or improper joint mechanics. Frame detection can be the ability to detect any point in time of the movement. Frame detection can be broader and encompass phase detection. Frame detection can be when the user chooses a particular frame the user would like the computer system to automatically detect. Phase detection can be the detection of specific frames that relate to the prior established/researched phases of that particular movement.

The computer system can detect and automatically produce a specific frame based on the phase of the movement and/or the results of the angle or point detection. Frame detection can be based on the phase of the movement including the computer system detecting a phase of gait, running or athletic movement and then displays that frame. For example, in gait and running examples, the computer system detects initial contact (when the foot first touches the ground) and displays that frame, and detects toe off (e.g., when the foot is about to leave the ground) and displays that frame. The displayed frame also carries/transfers and has the option to display the corresponding kinematic data with it. In another example, for athletic movements, such as running and cutting or deceleration, the computer system detects the exact moment when the athlete is making the transition from running straight to cutting to a side, and detects the exact moment when the athlete is making the transition from running straight to running backward.

Phase detection and frame detection can also be based on the results of the angle or point detection. Point detection is the ability to recognize and track any specific point on the video/image, for example, the center of knee cap. The computer system can detect the frame that has a specific parameter for the joint angles. Examples of phase detection in running include detection of initial contact, midstance and toe off. Examples of frame detection in running include maximum knee flexion and maximum tibial angle. With other movements such as throwing mechanics, serving mechanics, squatting mechanics etc, the system detects/finds each have phase detection and frame detection. Frame detection and phase detection can also occur with clinically validated tests and measure such as the Functional Movement Screen (FMS)™.

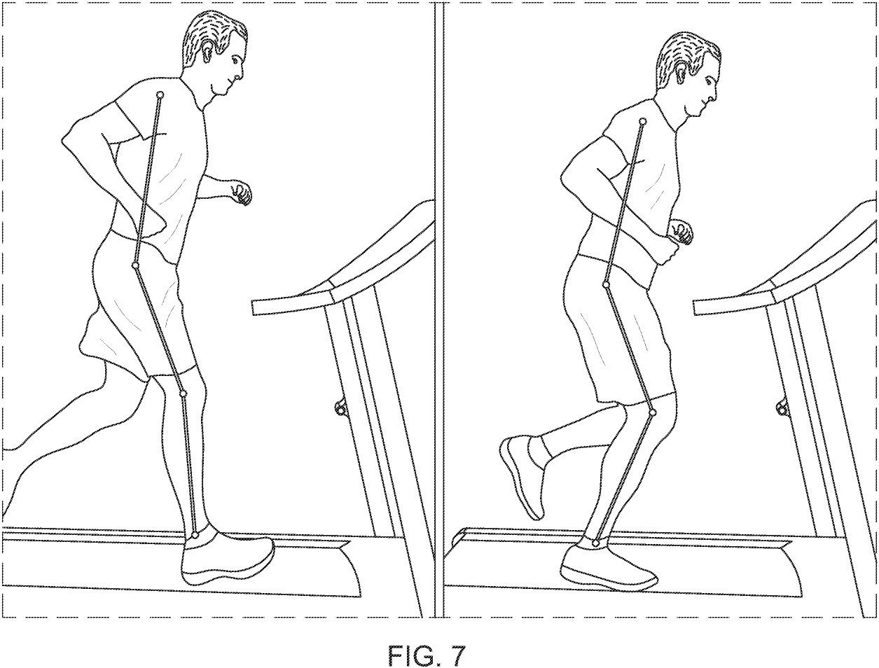

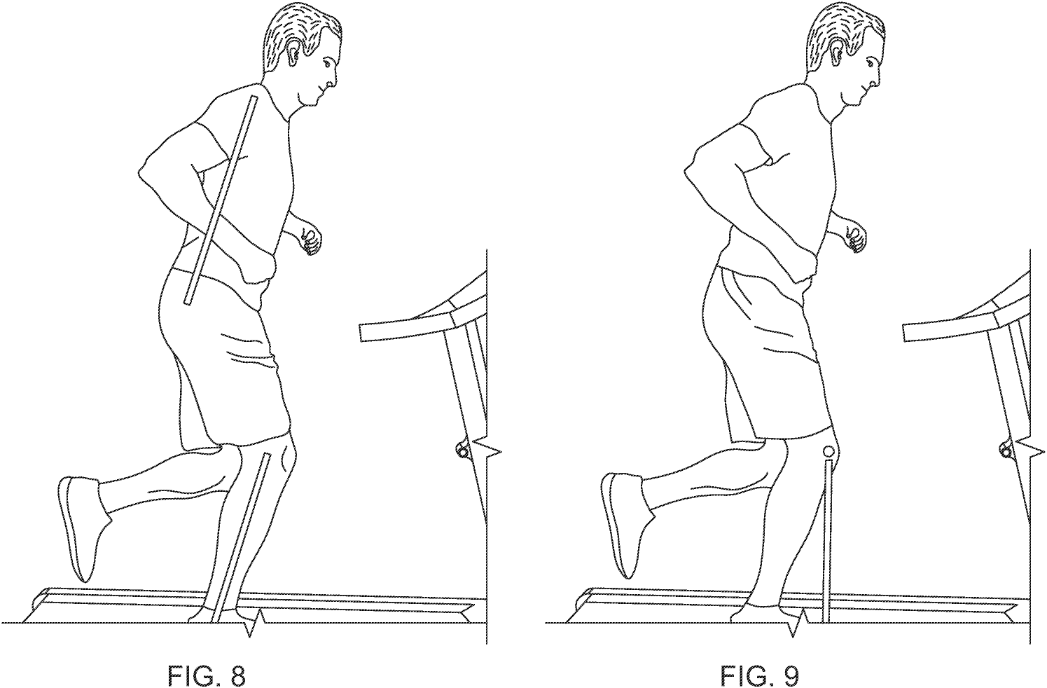

With respect to the comparison of data performed by the computer system, in some instances, the data values need to be compared with each other (rather than to normative values). These comparisons may include (1) comparing the same joint angle at two moments in time (phases). E.g., knee, hip and ankle excursion (the difference between the value of an angle in one phase compared to the value of that same angle in another phase) (e.g. FIG. 7), (2) comparing two different angles at the same moment in time (e.g., trunk angle vs tibial angle) (e.g., FIG. 8, and (3) comparing position of two points in space (e.g., compare knee to the foot/ ankle to see if knee is in front of toes) (e.g., FIG. 9) - (this can also be applicable for cross over sign). See remaining figures for examples.

Regarding analysis of specific calculations and/or metrics the computer system can measure, current technology requires separate extra hardware in addition to the software to capture the aforementioned kinematic and biomechanics data. The computer system disclosed herein does not require such separation. The software can perform calculations/manipulations of the data after the data is captured. The software can be added to any other hardware device with a camera system to do the capturing of the data. Then the software can perform calculations/manipulations of the data after the initial data is captured. Examples of specific calculations that the software can derive from this captured data may include shock absorption quantification (active or passive), shock absorption rating (this would put a numeric value on it and suggest if the force is absorbed more through the joints or more through the muscles), and estimation of impact force. Other examples include understanding if this is hip biased movement versus knee biased movement, prediction of loading rate, prediction of ground reaction force (GRF), and speed of force generation.

Movement determination/classification can include mobility, strength, coordination, or be based on the movement impairment. Movement determination/classification can also include insight into specific musculoskeletal or neurological conditions the subject presents with. Or which musculoskeletal conditions the subject is susceptible to as a result of the movement determination/classification they are exhibiting. Impairment ranking can list out movement impairments in order of their greatest severity or concern. A list of hypothesis’ as to the cause of the movement impairment can also be generated. For impairment ranking for hypothesis list and/or prediction of injuries, based on the results of the motion analysis data, the subjective/history input, demographics and other inputted data the computer system can predict what the determination/classification is.

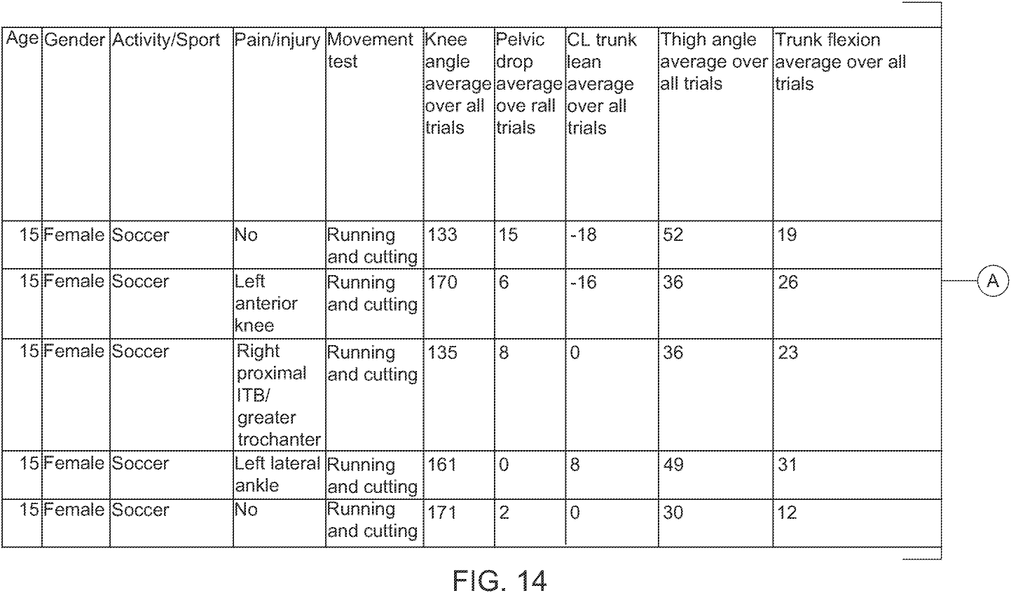

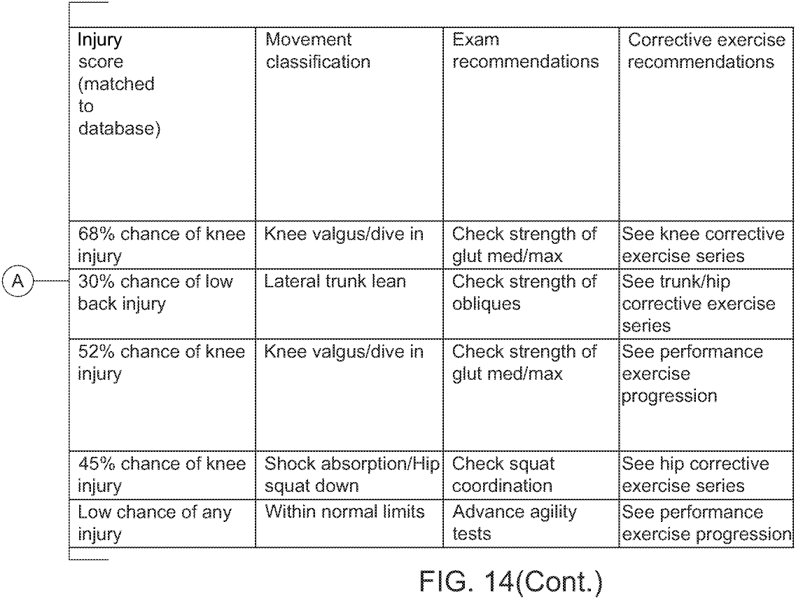

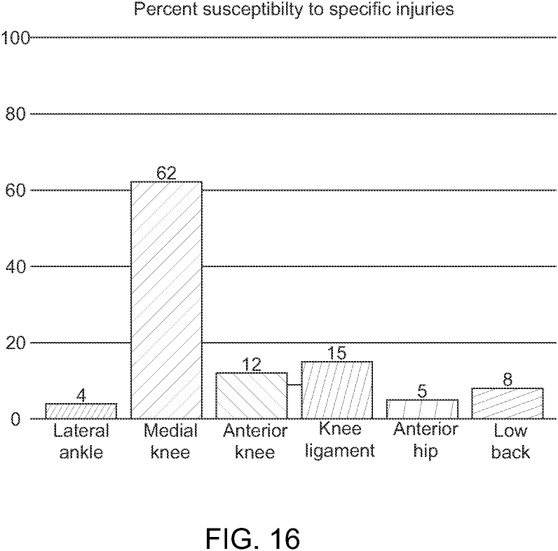

Here is an example of how the movement classification/determination and mpairment findings can predict and dictate susceptibility to specific musculoskeletal problems (e.g., excessive femoral adduction plus crossover sign plus pelvic drop equals pressure over the greater trochanter. This pressure can lead to pain at the trochanteric bursa). The user interface (UI) can display various musculoskeletal problems as a percentage that the client/patient is likely to be susceptible to a specific injury (e.g., 40% Increased likelihood of an anterior knee injury; 25% increased likelihood of lateral ankle injury)(e.g., as shown in FIGS. 14 and 16), by using normative values from current research and expert opinion, and/or the other users on the system (using, e.g., data analysis such as machine learning). This verifies the main key impairments as they relate to specific classifications / determinations.

In addition to impairment detection the computer system can suggest which muscles may or may not be activated, and the amount/level to which the muscle is activated.

Other recommendations for display can include (1) Injury prediction/susceptibility (even if symptoms are not present yet): type and severity. For example, “Subject exhibits quad dominance which can increase your risk for PFPS (retropatellar), quad tendinitis (proximal and or distal), and knee joint pain (intra-articular)”; (2) Practitioner subjective and/or objective/physical exam recommendations or client/subject/patient self-guided exam. For example, the computer system will provide recommendations to the practitioner based on data from various sources such as [cite one of the diagrams or figures] the client/patient intake form, practitioner taking a history, and video movement analysis. For example if the subject/client/patient has a history of a hamstring injury, the computer system may display recommendations to: 1. Check for contralateral hip flexor tightness as this can cause an anterior pelvic tilt, 2. Check for strength/activation of the gluteus maximus as this can contribute to hamstring overuse, 3. The cause of the impairment and areas of the body that are susceptible to stress, strain, and injury as a result of each impairment; 4. Ranking of each impairment based on percent likelihood this impairment is contributing to this condition as well as the severity of the impairment. This can help the clinician/practitioner guide treatment. For example, in order to determine which movement impairment is the highest relevance, the computer system will use severity of impairment as a guide. For subjects with symptoms, the computer system tells the user which impairment is the biggest cause of their symptoms. For subjects without symptoms, the computer system ranks which impairments are putting them at the highest risk for specific types of injuries. For example impairments with larger aberrant numeric values of joint angles might dictate the focus of the treatment); 5. practitioner guided treatment, and 6. client guided treatment.

For practitioner guided treatment, a combination of questions can be posed to the practitioner and/or the client in the form of text, check boxes or a visualization such as a body chart. Client questions may appear on an intake form answered prior to the motion analysis. Practitioner questions may appear before, during or after motion analysis. These questions can be bypassed. The resulting input from the practitioner and client will be inputted with the results of the motion analysis videos, frames and other data to allow the computer system to analyze. The computer system can analyze the results and give an output of suggestions for the interpretation of that data. These suggestions can include but not limited to the cause of the movement impairment, ranking severity of movement impairment, suggestions for further assessment, suggestions for corrective exercises, suggestions for products, suggestions for treatment and how to change that movement impairment.

For example, if back view knee valgus is detected, the computer system prompts the user with the following questions: (1) Location of pain, (2) Symptoms the client/patient has etc. This input can be combined with the input from the client/patient beforehand on the intake form where the client/patient can be prompted with questions such as (1) age, (2) gender, (3) weight, (4) height etc. Additionally, this data can also be obtained from integration with personal devices that collect data such as running distance, heart rate etc. An API can allow for integration with this computer system. The practitioner is provided the ability to override the input and manually input their current findings.

In this aforementioned example, the computer system combines manually inputted data from the practitioner, from the intake form from the client, the automated angles and health and athletic data from other platforms and applications in order to produce possible causes and suggested exercises, products and treatment.

The computer system can use a method of data analysis such as machine learning to develop algorithms that tell the user which treatment option is most likely to help the most and predict which injuries users are most susceptible to. The computer system can rank which impairments are the highest priority and need to be treated first.

This data analysis can be used for predicting onset of future injuries in order to provide preventative measures. This can be done by comparing to other users who have had similar analyses or by gathering other data. These analyses combined along with their other data (e.g. manually inputted text data, demographic information, clinical findings, etc.) as well as integrations with other software and devices that allow for health and performance data collection such as heart rate, speed, running distance, etc. creates new data insights. This data forms a database of motion analysis and kinematic data and correlates the motion analysis to known outcomes such as whether that individual experienced pain or injury. For example, a practitioner the user collects motion analysis data and kinematic data on an individual. Time series data is collected by repeatedly performing the motion analysis on one individual over time as well as collecting data on specific joint angles with various individuals. The subjective history such as pain scale, pain intensity, location of symptoms etc is correlated to the motion analysis and kinematic data. Through data analysis such as machine learning, predictive patterns emerge over time. This prediction can be displayed for the user. In one example, vertical trunk during initial contact phase of running for males 35-55 has been shown to lead to a 68% chance of anterior knee pain. In another example, based on the data obtained compared to a database, this runner/athlete has a 32% increased susceptibility to an ankle injury and 14% increased susceptibility to a knee injury. Furthermore, motion analysis data can be stored and can be segmented/divided up by a population such as runners, athletes and patients or segmented based on demographic information such as height and weight etc or any data combination thereof. Then the system can perform a normalization of the motion analysis data for each individual based on values of the motion analysis data within their particular population. This then generates a profile comprising of motion analysis data for each individual with respect to their particular population or demographics. The practitioner/user has the ability to manipulate this segmentation of the data to allow for predictions on a diverse group of people, athlete and runners.

For subject/client/patient guided analysis, the computer system can provide instructions and recommendations for how to capture the best video data, image data and other forms of data. These instructions and recommendations can be based on previous results of the client. For client guided treatment, the computer system can provide exercise recommendations to the client based on client subjective input to questions posed by the computer system, and self-video movement analysis.

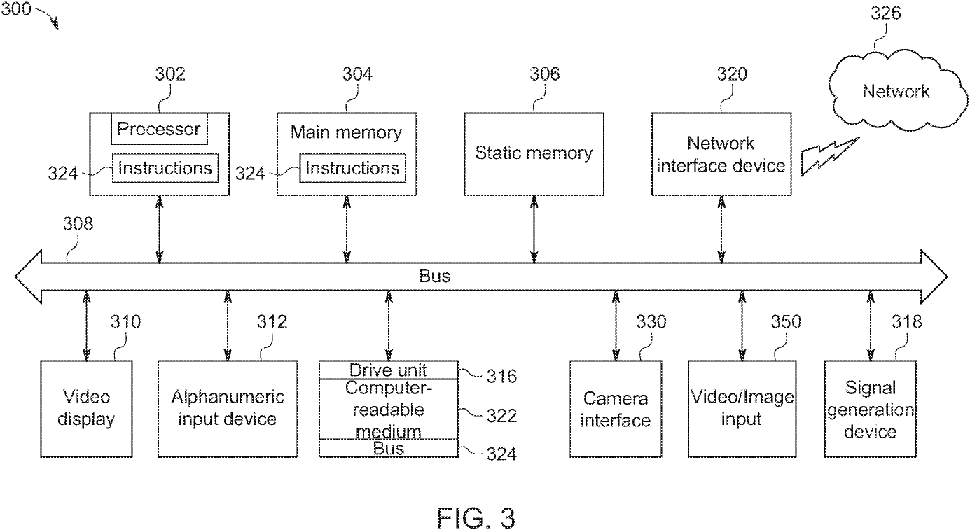

FIG. 3 shows a diagrammatic representation of a machine in the example form of a machine or computer system 300 within which a set of instructions 304 may be executed causing the machine to perform any one or more of the methodologies discussed herein. In alternative embodiments, the machine operates as a standalone device or may be connected (e.g., networked) to other machines. In a networked deployment, the machine may operate in the capacity of a server 102 or a client machine 101 in a server-client network environment, or as a peer machine in a peer-to-peer (or distributed) network environment. The machine may be a personal computer (PC), a tablet PC, a set-top box (STB), a personal digital assistant (PDA), a cellular telephone, a web appliance, a network router, switch or bridge, or any machine capable of executing a set of instructions 304 (sequential or otherwise) that specify actions to be taken by that machine. Further, while only a single machine is illustrated, the term “machine’ shall also be taken to include any collection of machines that individually or jointly execute a set (or multiple sets) of instructions 304 to perform any one or more of the methodologies discussed herein.

The example computer system 300 includes a processor 302 (e.g., a central processing unit (CPU), a graphics processing unit (GPU), or both), a main memory 304, and a static memory 306, which communicate with each other via a bus 308. The computer system 300 may further include a video display unit 310 (e.g., a liquid crystal display (LCD) or a cathode ray tube (CRT)). The computer system 300 also includes an alphanumeric input device 312 (e.g., a key board), a UI navigation device 314 (e.g., a mouse or pad), a drive unit 316, a signal generation device 318 (e.g., a speaker), a network interface device 320, a camera interface 330 capable of receiving captured videos and/or still images, and a video/image input 350 source capable of receiving input videos and/or still images.

The drive unit 316 includes a computer-readable medium 322 on which is stored one or more sets of data structures and instructions 324 (e.g., software) embodying or utilized by any one or more of the methodologies or functions described herein. The instructions 324 may also reside, completely or at least partially, within the main memory 304 or within the processor 302 during execution thereof by the computer system 300, with the main memory 304 and the processor 302 also constituting machine-readable media.

The instructions 324 may further be transmitted or received over a network 326 via the network interface device 320 utilizing any one of a number of well-known transfer protocols (e.g., HTTP).

While the computer-readable medium 322 is shown in an example embodiment to be a single medium, the term “computer-readable medium’ should be taken to include a single medium or multiple media (e.g., a centralized or distributed database, and/or associated caches and servers) that store the one or more sets of instructions 324. The term “computer-readable medium’ shall also be taken to include any medium that is capable of storing, encoding, or carrying a set of instructions 324 for execution by the machine that cause the machine to perform any one or more of the methodologies of the present disclosure, or that is capable of storing, encoding, or carrying data structures utilized by or associated with such a set of instructions 324. The term “computer-readable medium’ shall, accordingly, be taken to include, but not be limited to, solid-state memories, optical media, and magnetic media.

Furthermore, the machine-readable medium is non transitory in that it does not embody a propagating signal. However, labeling the tangible machine-readable medium “non-transitory’ should not be construed to mean that the medium is incapable of movement—the medium should be considered as being transportable from one physical location to another. Additionally, since the machine-readable medium is tangible, the medium may be considered to be a machine readable device.

Although an embodiment has been described with reference to specific example embodiments, it will be evident that various modifications and changes may be made to these embodiments without departing from the broader spirit and scope of the present disclosure. Accordingly, the specification and drawings are to be regarded in an illustrative rather than a restrictive sense. The accompanying drawings that form a part hereof show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

As used herein, the term “or may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present invention. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present invention as represented by the appended claims. The specification and drawings are, accordingly, to be regarded in an illustrative rather than a restrictive sense.

Such embodiments of the inventive subject matter may be referred to herein, individually or collectively, by the term “invention’ merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Some portions of the preceding detailed descriptions have been presented in terms of algorithms. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a mobile device, or similar electronic device, that manipulates and transforms data represented as physical (electronic) quantities within the system’s registers and memories into other data similarly represented as physical quantities within the system memories or registers or other such information storage, transmission or display devices.

The processes and blocks described herein are not limited to the specific examples described and are not limited to the specific orders used as examples herein. Rather, any of the processing blocks may be re-ordered, combined or removed, performed in parallel or in serial, as necessary, to achieve the results set forth above. The processing blocks associated with implementing the system may be performed by one or more programmable processors executing one or more computer programs stored on a non-transitory computer readable storage medium to perform the functions of the system. All or part of the system may be implemented as, special purpose logic circuitry (e.g., an FPGA (field-programmable gate array) and/or an ASIC (application-specific integrated circuit)). All or part of the system may be implemented using electronic hardware circuitry that include electronic devices such as, for example, at least one of a processor, a memory, a programmable logic device or a logic gate. Further, processes can be implemented in any combination hardware devices and software components.

In addition, in the foregoing Detailed Description, it can be seen that various features are grouped together in a single embodiment for the purpose of streamlining the dis closure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A computer-implemented method comprising:
obtaining, by a computer system having a display screen coupled to the computer system, movement analysis data captured via a camera coupled to the computer system or input into the computer system, the movement analysis data comprising at least one critical phase or frame and/or instance in time of body movement;
detecting, by the computer system, a movement impairment within one of the at least one critical phase or frame and/or instance in time of body movement, based at least on the obtained movement analysis data;
detecting, by the computer system, motion analysis on videos that were previously captured by overlaying the data on the video and/or frames; and
displaying, by the computer system on the display screen, information related to the detected movement impairment.

2. The method of claim 1 wherein the movement analysis data is captured via the camera for a plurality of moving bodies, and a movement impairment is detected for each of the plurality of moving bodies.

3. The method of claim 1 wherein the movement analysis data is captured using markerless tracking.

4. The method of claim 1 wherein the step of detecting further comprises one or more of the following:
detecting anatomical landmarks;
detecting any point the user chooses;
detecting joint angles;
detecting distance between a plurality of points;
detecting a direction in which a body is moving;
detecting a running cadence and stride length of the moving body;
detecting a center of mass displacement of the moving body;
detecting other kinematic data the user selects; and
detecting and labeling a type of joint or body part of the static or moving body.

5. The method of claim 1 wherein the step of detecting further comprises the following:
modifiable parameters for the user for point detection and landmark detection;
modifiable parameters by the computer system for point detection and landmark detection; and
modifiable parameters as to which phase(s) and/or frame(s) are detected.

6. The method of claim 1 wherein the computer system further comprises a server, and the method further comprises:
transmitting the obtained movement analysis data to the server; and
performing said detection in near real-time at the server.

7. The method of claim 6 wherein the obtained movement analysis data is capable of being integrated with other platforms.

8. The method of claim 1 wherein said detecting is performed at the computer system connected to the display screen in real-time.

9. The method of claim 1 wherein said detecting comprises performing one or more of the following:
comparing processed movement analysis data and normative values;
comparing processed movement analysis data and historical data;
comparing processed movement analysis data with itself; and
using manually input text data.

10. The method of claim 1 further comprising predicting a likelihood that a specific injury will occur based at least on analysis of the movement data.

11. The method of claim 1 wherein said displaying comprises performing one or more of the following:
displaying a classification or determination of the impairment;
displaying one or more highlighted sections of the movement analysis data which are deemed red flags or outliers;
displaying exam recommendations; and
displaying impairment corrections and/or treatment.

12. The method of claim 10 wherein displaying exam recommendations comprises providing an impairment ranking for a diagnostic hypothesis list and/or prediction of an injury.

13. The method of claim 1 further comprising:
detecting at least one of critical phases of specific movements or frames specified by the user within the movement analysis data.

14. The method of claim 13 further comprising detecting a specific frame within a critical phase of the body movement or frames specified by the user based on angle or point detection.

15. The method of claim 1 further comprising:
creating a central data repository and conducting data analysis such as machine learning to create predictions.

16. The method of claim 2 further comprising gathering data for each individual by identifying individual people in the frame/video so a database of each individual person’s data can be collected and added to over time and displayed in a respective portal.

17. A system comprising:
a processor;
a user interface coupled to the processor, the user interface comprising an input device, a camera, and a display screen; and
memory coupled to the processor and storing instructions that, when executed by the processor, cause the system to perform operations comprising:
obtaining movement analysis data captured via the camera coupled to the user interface or input into the user interface, the movement analysis data comprising at least one critical phase of body movement or frames specified by the user;
detecting a movement impairment within one of the at least one critical phase of body movement or frames specified by the user, based at least on the obtained movement analysis data; and
displaying on the display screen information related to the detected movement impairment.

18. A non-transitory computer-readable medium storing instructions that, when executed by a computer system, cause the computer system to:
obtain, by a computer system having a display screen coupled to the computer system, movement analysis data captured via a camera coupled to the computer system or input into the computer system, the movement analysis data comprising at least one critical phase or frame and/or instance in time of body movement;
detect, by the computer system, a movement impairment within one of the at least one critical phase or frame and/or instance in time of body movement, based at least on the obtained movement analysis data;
detect, by the computer system, motion analysis on videos that were previously captured by overlaying the data on the video and/or frames; and
display, by the computer system on the display screen, information related to the detected movement impairment.
